(19) United States
(12) Patent Application Publication
(10) Pub. No.: US 2015/0157277 A1
(43) Pub. Date: Jun. 11, 2015

(54) MAGNETIC RESONANCE IMAGING APPARATUS AND MAGNETIC RESONANCE IMAGING METHOD

(71) Applicant: HITACHI MEDICAL CORPORATION, Tokyo (JP)

(72) Inventors: Tomohiro Goto, Tokyo (JP); Masahiro Takizawa, Tokyo (JP)

(21) Appl. No.: 14/405,035

(22) PCT Filed: Jul. 24, 2013
(86) PCT No.: PCT/JP2013/070015

§ 371 Date: Dec. 2, 2014

(30) Foreign Application Priority Data
Aug. 13, 2012 (JP) 2012-179360

(51) Int. Cl. A61B 5/00; A61B 5/11; A61B 5/055

(52) U.S. Cl. CPC A61B 5/7285; A61B 5/055; A61B 5/1128

(57) ABSTRACT

In order to respond to positional changes of body motion, such as respiratory motion, in various directions and to prevent an increase in the imaging time due to acquisition of body motion information or the occurrence of dead time in the measurement, a control unit of an MRI apparatus acquires association information in which body motion information detected by an external monitor, such as a pressure sensor for monitoring the movement of an object to be examined, and body motion information measured from an NMR signal by the navigator sequence are associated with each other in advance. During imaging, body motion information from the navigator is estimated using body motion information detected by an external monitor mounted on the object to be examined and the association information acquired in advance, and control, such as performing gating imaging or correcting the imaging slice position based on the estimated body motion position, is performed.

### TECHNICAL FIELD

The present invention relates to a magnetic resonance imaging (MRI) apparatus that measures a nuclear magnetic resonance (hereinafter, referred to as “NMR”) signal from hydrogen, phosphor, or the like in an object and images nuclear density distribution, relaxation time distribution, or the like. In particular, the present invention relates to an MRI apparatus that performs imaging in consideration of body motion of an object to be examined.

### BACKGROUND ART

In an examination using an MRI apparatus, artifacts due to respiratory motion often become a problem. Breath-hold imaging is used as the simplest method, and this is widely used clinically. However, there is a limitation that the breath-hold imaging cannot be applicable for an object who has difficulty holding their breath or that a single imaging time is limited to a period for which it is possible for the object to hold their breath (about 15 seconds at the longest).

As a method of suppressing respiratory motion artifacts without breath-holding, there is a method using an external monitor (PTL 1). This is a method of suppressing the occurrence of artifacts by monitoring of the respiratory motion of the abdominal wall with a pressure sensor or the like to acquire data of only a specific breathing phase. This method is advantageous in that the respiratory status can always be monitored even during imaging since a sensor is attached to an object.

In addition, as another method of suppressing the respiratory motion artifacts without breath-holding, there is a navigator echo method (PTL 2). The navigator echo method is a method of acquiring additional echoes for monitoring respiratory motion separately from the acquisition of image data and performing gating and position correction using the respiratory motion information acquired from the echoes. This is a highly versatile method since it is possible to monitor the positional change of a certain part (for example, movement of a diaphragm in the H-F direction), compared with a method using an external monitor.

### CITATION LIST

### Patent Literature

[PTL 1] JP-A-2008-148806

[PTL 2] JP-A-2008-154887

### SUMMARY OF INVENTION

### Technical Problem

In the method using an external monitor, there is a disadvantage that the versatility is low, for example, only movement in a specific direction (in general, vertical movement of the abdominal wall) of the respiratory motion can be monitored. For example, not only vertical movement but also movement in a head-foot direction (hereinafter, abbreviated as an H-F direction) is included in the respiratory motion. However, it is not possible to perform imaging in a state where the slice position follows the movement in the H-F direction using a pressure sensor fixed to the abdominal wall.

In the navigator echo method, apart from the main imaging, a sequence execution time for acquiring the navigator echo is required. For this reason, dead time occurs during measurement. For example, in the case of acquiring an image in the entire cardiac cycle as in the cine imaging of the heart, it is not possible to acquire an image in the cardiac phase of the navigator sequence execution part.

Therefore, it is an object of the present invention to respond to positional changes of body motion, such as respiratory motion, in various directions and to prevent an increase in the imaging time due to acquisition of body motion information or the occurrence of dead time in the measurement.

### Solution to Problem

In order to solve the aforementioned problem, a magnetic resonance imaging apparatus of the present invention uses body motion information from at least two body motion monitors. In addition, association information is created by associating the body motion information from a plurality of body motion monitors, and imaging is controlled using the association information and body motion information from one of the body motion monitors during the imaging. The imaging control may be either gating for controlling the timing at which an NMR signal is acquired or the correction of the slice position where the NMR signal is acquired.

### Advantageous Effects of Invention

According to the present invention, since the information from a plurality of body motion monitors is used, it is also possible to respond to positional changes in different directions. In addition, since the association information of a plurality of pieces of body motion information is used, body motion information of other body motion monitors can be estimated by using the body motion information from one body motion monitor. Therefore, it is also possible to respond to positional changes in different directions similar to the case where a plurality of body motion monitors are used. For this reason, since a navigator sequence during imaging can be eliminated, it is possible to prevent an increase in the imaging time due to acquisition of body motion information or the occurrence of dead time during measurement.

### BRIEF DESCRIPTION OF DRAWINGS

FIG. 1(a) is a block diagram showing the overall configuration of an MRI apparatus to which the present invention is applied, and FIG. 1(b) is a functional block diagram of a control unit.

FIG. 2 is a flowchart showing the operation of an MRI apparatus of a first embodiment.

FIG. 3 is a flowchart of a pre-scan part of the first embodiment.

FIG. 4 is a diagram showing an example of the navigator sequence of a pre-scan that the MRI apparatus of the first embodiment includes.

FIGS. 5(a) and 5(b) are diagrams explaining the displacement detected by the navigator sequence and the displacement detected by a pressure sensor, and FIG. 5(c) is a diagram explaining the association of displacement.

FIGS. 6(a) and 6(b) are diagrams showing an example of association information (fitting function).

FIG. 7(a) is a diagram explaining the slice correction using the fitting function, and FIG. 7(b) is a diagram explaining the gating using the fitting function.

FIG. 8 is a diagram explaining the effects of the first embodiment.

FIG. 9 is a flowchart showing the operation of an MRI apparatus of a second embodiment.

FIG. 10 is a diagram showing an example of the displacement measured at different times by body motion monitors.

FIG. 11 is a diagram explaining the amount of imaging slice position correction in a third embodiment.

FIG. 12 is a diagram showing an example of measuring the displacement in a plurality of regions in a fourth embodiment, where FIG. 12(a) is a diagram showing the COR plane, FIG. 12(b) is a diagram showing the Ax (axial) plane, and FIG. 12(c) is a diagram showing the relationship between the displacement of a plurality of regions and the displacement detected by the external monitor.

FIG. 13 is a diagram showing the association information (fitting function) of each displacement acquired in FIG. 12(c).

### DESCRIPTION OF EMBODIMENTS

First, the outline of an MRI apparatus of the present invention will be described. An MRI apparatus includes: an imaging unit including a static magnetic field magnet, a gradient magnetic field generation unit, a high-frequency magnetic field transmission unit, and a nuclear magnetic resonance signal receiving unit; a signal processing unit that performs processing including image reconstruction using a nuclear magnetic resonance signal received by the receiving unit; and a control unit that controls the imaging unit and the signal processing unit.

The control unit includes a body motion processing unit that receives body motion information from a plurality of body motion monitors that monitor a body motion of an object to be examined and associates a plurality of motions detected by the plurality of body motion monitors, and controls the imaging unit using body motion information detected by one of the plurality of body motion monitors and association information calculated by the body motion processing unit.

For example, the control unit estimates body motion information of body motion monitors other than the one body motion monitor using the body motion information detected by one of the plurality of body motion monitors and the association information calculated by the body motion processing unit, and controls the imaging unit using the estimated body motion information.

At least one of the plurality of body motion monitors can be an internal monitor that detects a body motion using the nuclear magnetic resonance signal received by the receiving unit, and at least one of the plurality of body motion monitors can be an external monitor that detects a movement of the object to be examined using a physical method. The direction of the movement detected by the internal monitor and the direction of the movement detected by the external monitor may be different or may be the same.

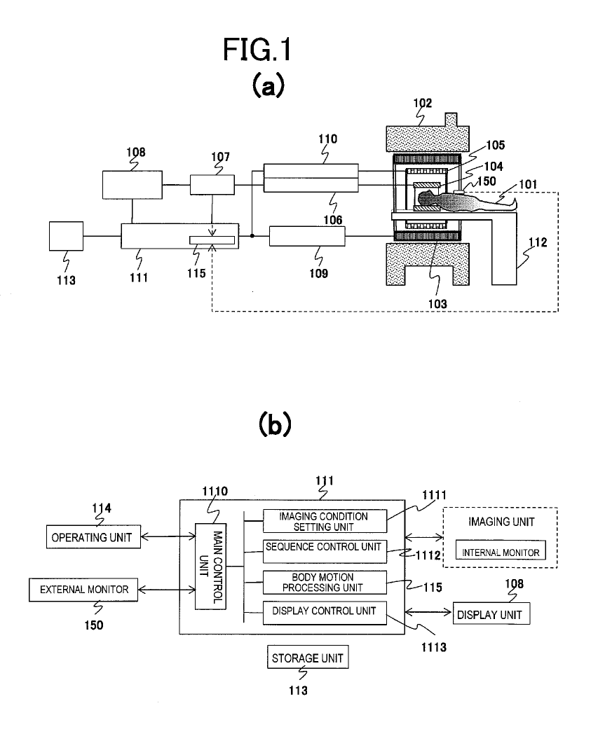

Hereinafter, an embodiment of the present invention will be described with reference to the diagrams. FIG. 1(a) is a block diagram showing the configuration of an MRI apparatus of the present embodiment. The MRI apparatus includes a magnet 102 for generating a static magnetic field in space (imaging space) where an object 101 is placed, a gradient magnetic field coil 103 for generating a gradient magnetic field in the imaging space, an RF coil 104 for irradiating a high-frequency magnetic field to a predetermined region of the object placed in the imaging space, and an RF probe 105 for detecting an NMR signal generated from the object 101. In general, the object 101 is inserted into the imaging space in a state of lying on a bed 112, and imaging is performed.

The gradient magnetic field coil 103 is formed by gradient magnetic field coils in three axial directions of X, Y, and Z, and generates a gradient magnetic field according to a signal from a gradient magnetic field power source 109. The RF coil 104 generates a high-frequency magnetic field according to a signal of an RF transmission unit 110. The signal of the RF probe 105 is detected by a signal detection unit 106, and is subjected to signal processing by a signal processing unit 107 or is converted into an image signal by calculation. An image is displayed on a display unit 108. The gradient magnetic field power source 109, the RF transmission unit 110, and the signal detection unit 106 are controlled by a control unit 111. The time chart of control is generally called a pulse sequence, and various pulse sequences are prepared according to the imaging method and are stored as a program in the control unit 111. During imaging, the pulse sequence corresponding to the purpose is read and executed. The control unit 111 includes a storage unit 113 for storing parameters or the like required for imaging and an operating unit 114 that is used when a user inputs information required for control.

The MRI apparatus of the present invention acquires body motion information from a plurality of body motion monitors to control the imaging. More specifically, a plurality of pieces of body motion information are received from a plurality of body motion monitors for monitoring the body motion of the object, and the plurality of pieces of body motion information detected by the plurality of body motion monitors are associated with each other. In addition, imaging is controlled using the association information and the body motion information detected by one of the plurality of body motion monitors. Therefore, a body motion processing unit 115 that associates a plurality of pieces of body motion information detected by the plurality of body motion monitors is provided. The plurality of body motion monitors may be external monitors, or may be a combination of an external monitor and an internal monitor. The external monitor is a body motion monitor that is physically separated from the MRI apparatus. For example, it is possible to use a pressure sensor or bellows fixed to the abdominal wall or a three-dimensional detector for detecting the position of the abdominal wall or the like.

FIG. 1(a) shows a state where a body motion sensor 150 is attached to the abdomen of the object 101 as an example. The position information detected by the external monitor 150 is input to the body motion processing unit 115 through a signal line and an external input terminal. The internal monitor is means for detecting an object position using the NMR signal detected by the signal detection unit 106 of the MRI apparatus. Specifically, a signal collection pulse sequence, such as a navigator sequence, is included. In the pulse sequence, such as a navigator sequence, it is possible to acquire the NMR signal from an arbitrary region by changing the conditions of the gradient magnetic field, and it is possible to detect the positional change of the region from the NMR signal.

The relationship between the control unit 111 and an internal monitor and the external monitor 150 when the control unit 111 shown in FIG. 1(a) includes the external monitor 150 is shown in a functional block diagram of FIG. 1(b). In this diagram, for an imaging unit, a portion excluding the display unit 108, the control unit 111, and the storage unit 113 shown in FIG. 1(a) is collectively expressed as an imaging unit. In addition, as described above, the internal monitor is means for detecting the object position using the NMR signal detected by the signal detection unit 106 of the MRI apparatus, and is described as being included in the imaging unit. The control unit 111 includes not only a main control unit 1110 but also an imaging condition setting unit 1111, a sequence control unit 1112, the body motion processing unit 115, a display control unit 1113, and the like. Functions of these units will be described together with their operations in each of the following embodiments.

Based on the outline of the MRI apparatus described above, each embodiment of the present invention will be described focusing on the operations of the control unit 111 and the body motion processing unit 115.

### First Embodiment

The MRI apparatus of the present embodiment is characterized in that a respiratory motion monitor (an aspect of the internal monitor) using a navigator echo and a respiratory motion monitor (an aspect of the external monitor) of the abdominal wall, such as a pressure sensor, are used as a plurality of body motion monitors.

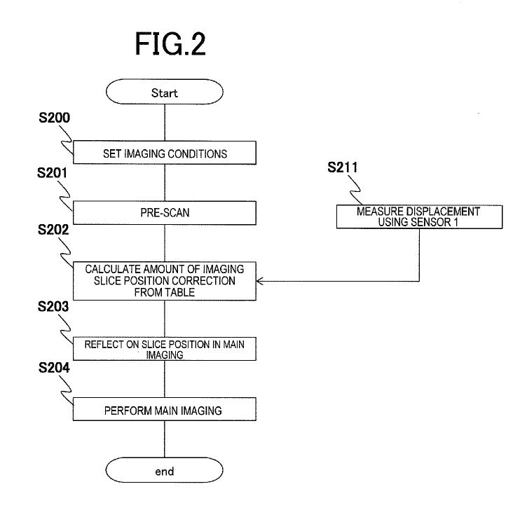

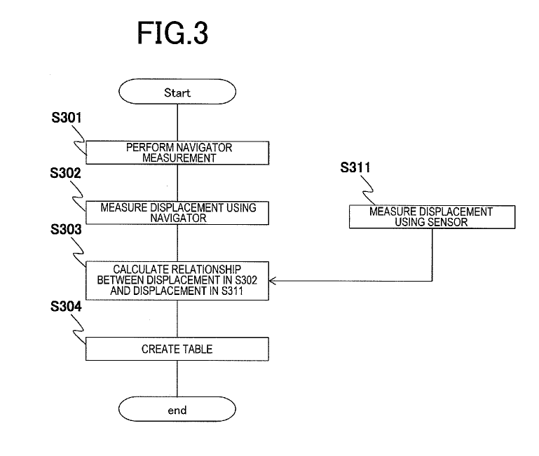

FIGS. 2 and 3 show the procedure of the imaging control performed by the control unit 111. FIG. 2 is a flowchart showing the procedure of the entire imaging process, and FIG. 3 is a flowchart showing a part of a pre-scan.

First, the imaging condition setting unit 1111 sets the imaging conditions (S200). Here, conditions related to the imaging region, such as a slice position (direction), a slice width, and a gate window, are set based on a scanogram (wide area image obtained by imaging the object with relatively low resolution prior to the examination), and parameters of the pulse sequence used in main imaging, for example, echo time (TE), repetition time (TR), and the number of times of addition, are set. The gate window is for setting the signal-collectable body motion width when performing gating imaging using a navigator in units of mm or pixel, and is appropriately set according to the purpose of imaging (for example, a high-quality image or time resolution priority). These conditions and parameters are set in the control unit 111 through input means. Although the slice direction can be set arbitrarily, explanation herein will be given on the assumption that the slice direction is set to the H-F direction.

When a position to be imaged and a pulse sequence for imaging are determined, the sequence control unit 1112 performs a pre-scan for acquiring the association information (hereinafter, also referred to as a table) of a plurality of body motion sensors (FIG. 2: S201). The creation of the table may be performed as a measurement that is separated from the flow of main imaging, or may be performed as a pre-scan before the main imaging. In the flow shown in FIG. 2, a case is shown in which the creation of the table is performed as a pre-scan before the main imaging.

In the pre-scan, only the navigator sequence is continuously executed (FIG. 3: S301). As the navigator sequence, it is possible to use a known pulse sequence for locally exciting only a part under respiratory motion.

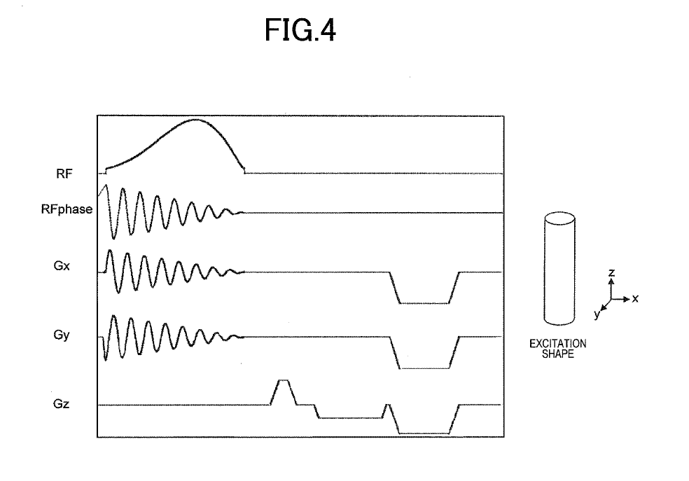

FIG. 4 shows an example of the navigator sequence. In this pulse sequence, at the time of excitation using an RF pulse, gradient magnetic fields Gx and Gy that vibrate in x and y directions are applied to excite a cylindrical region extending in a z direction. In the present embodiment, the z direction is a direction (H-F direction) parallel to the body axis of the object. Then, an NMR signal (not shown) is acquired by performing read-out in the z direction (Gz) without applying phase encoding. This NMR signal is referred to as a navigator echo. The profile of the signal value is obtained by performing a Fourier transform of the navigator echo in the frequency direction. A plurality of profiles of different measurement times are obtained by repeating the measurement of such a navigator echo at predetermined time intervals. In general, the respiratory cycle is in the order of a few seconds. Therefore, the navigator sequence is executed at intervals of about several hundreds of milliseconds.

In addition, as a pulse sequence serving as an internal monitor, it is possible to adopt not only the pulse sequence shown in FIG. 4 but also a sequence for acquiring an echo signal by exciting a columnar region by selecting the slices in axial directions perpendicular to each other and various methods, such as a method of setting an ROI in a low-resolution image and tracking the displacement of a predetermined part, such as a diaphragm, in the ROI.

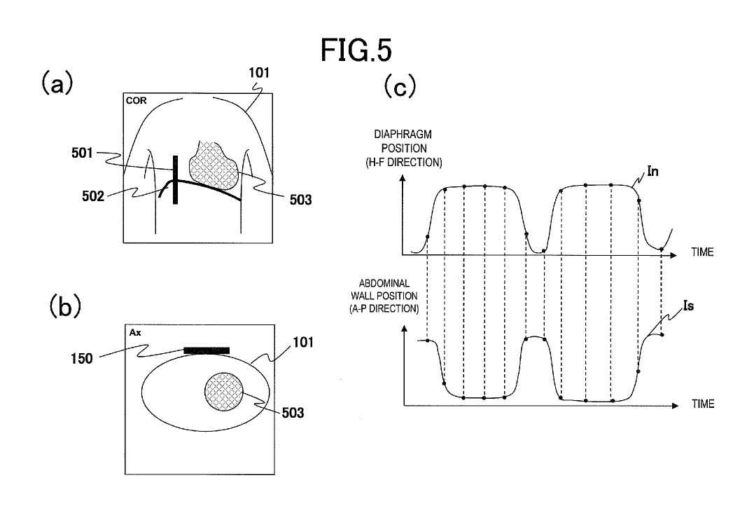

FIG. 5 shows the relationship between the respiratory motion and a region excited in the navigator sequence. As shown in FIG. 5(a), in the navigator sequence, a cylindrical region 501 that crosses a diaphragm 502 of the object 101 is excited. The position of the diaphragm 502 in the region 501 moves in the H-F direction according to the respiratory motion of a lung 503. Therefore, by tracking the position of the diaphragm 503 in a plurality of profiles, it is possible to monitor a respiratory displacement In in the H-F direction as shown in the upper graph in FIG. 5(c) (S302). In addition, instead of tracking the position of the diaphragm 503, it is also possible to track a positional change using a method, such as profile pattern matching. In this case, a region to be excited is not limited to the region that crosses the diaphragm. The respiratory displacement is calculated as a change in the relative value (unit is mm or pixel) with respect to the reference position (for example, an initial position at the start of measurement).

In parallel with the execution of the navigator sequence, a positional change (displacement) is tracked by a pressure sensor 150 (S311). As shown in FIG. 5(b), the pressure sensor 150 is mounted between a belt fixed to the object 101 and the abdominal wall, and is intended to track a pressure change due to vertical movement of the abdominal wall. The positional change detected by such a pressure sensor is a respiratory displacement Is in a vertical direction (A-P direction) perpendicular to the body axis of the object as shown in the lower graph in FIG. 5(c), and is detected as a change in a relative value (no units) with respect to the reference position (for example, an initial position) similar to the respiratory displacement In in the H-F direction.

In FIG. 5(c), the vertical axis indicates a position (relative value), and the horizontal axis indicates time. As shown in the diagram, both the respiratory displacements In and Is detected by two body motion monitors are based on the same respiratory motion, and the periods are the same.

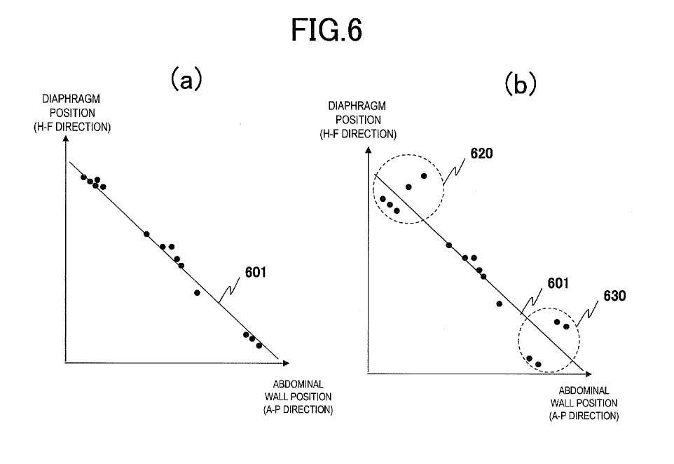

Then, the body motion processing unit 115 associates the respiratory displacement In obtained by the navigator sequence with the respiratory displacement Is obtained by the pressure sensor 150 (S303). The association of the respiratory displacements In and Is can be performed by calculating a function 601 by performing, for example, linear-function fitting for the distribution of the displacement shown in FIG. 6(a). A least square method or the like is generally used for the fitting.

For example, assuming that the position x in the A-P direction, which is detected by the pressure sensor 150, and the position z in the H-F direction, which is measured by the navigator sequence, at the same time are (x1, z1), (x2, z2), (x3, z3), . . . , (xn, zn), the straight line that fits the most is expressed by Expression (1).

$$\begin{matrix}[ Expression 1 ] & \\ Z = ax + b \\ a = \frac{n {\sum }_{k = 1}^{n} {x}_{k} {z}_{k} - {\sum }_{k = 1}^{n} {x}_{k} {\sum }_{k = 1}^{n} {z}_{k}}{n {\sum }_{k = 1}^{n} {x}_{k}^{2} - {( {\sum }_{k = 1}^{n} {x}_{k} )}^{2}} \\ b = \frac{{\sum }_{k = 1}^{n} {x}_{k}^{2} {\sum }_{k = 1}^{n} {z}_{k} - {\sum }_{k = 1}^{n} {x}_{k} {z}_{k} {\sum }_{k = 1}^{n} {x}_{k}}{n {\sum }_{k = 1}^{n} {x}_{k}^{2} - {( {\sum }_{k = 1}^{n} {x}_{k} )}^{2}} & ( 1 )\end{matrix}$$

The number of data points (n) is not particularly limited, but it is preferable that the number of data points (n) be equal to or greater than one period of the respiratory period so that the data of a plurality of periods is acquired.

In addition, although a case where the relationship between the respiratory displacements In and Is shown in FIG. 5(c) is the same in an inhale period and an exhale period is assumed in Expression (1), a respiratory period may be divided into an inhale period and an exhale period and a fitting function may be calculated for each of the inhale period and the exhale period when a possibility that the relationship between the respiratory displacements In and Is may be different in the inhale period and the exhale period is taken into consideration.

In addition, although the peak of the respiratory displacement In and the peak of the respiratory displacement Is are the same timing in the example shown in FIG. 5(c), the peaks may be shifted from each other. That is, a delay may occur. In this case, the delay causes variations shown in regions 620 and 630 in the distribution shown in FIG. 6(b). However, the offset or inclination of a fitting function 601 just changes by the amount of variation. Therefore, this case can be treated in the same manner as a case where there is no delay.

The fitting function showing the relationship between the respiratory displacement In and the respiratory displacement Is, which has been acquired as described above, is stored in the storage unit 113 as association information (table). The unit of the value stored in a table is mm or pixel. By the above processing, the pre-scan step S201 in FIG. 2 is ended (S304).

Next, main imaging is started. The main imaging continues from the pre-scan, and the position xi of the respiratory displacement (in the A-P direction) is detected from a body motion monitor 150 mounted on the object 101 and the result is input to the body motion processing unit 115 (S211). The body motion processing unit 115 estimates the position zi in a slice selection direction (H-F direction) using the detected position xi and the association information (the fitting function or the table) 601 between the respiratory displacement In and the respiratory displacement Is acquired in the pre-scan S201 (S202).

The detection of the respiratory displacement Is (position) by the pressure sensor 150 (S211) and the estimation of the position in the H-F direction using the same (S202) are continuously performed during the execution of the main imaging (S204). This is used in the control of the main imaging, specifically, in the correction of the slice position or gating. In the flow of FIG. 2, a case where slice position correction (S203) is performed is shown.

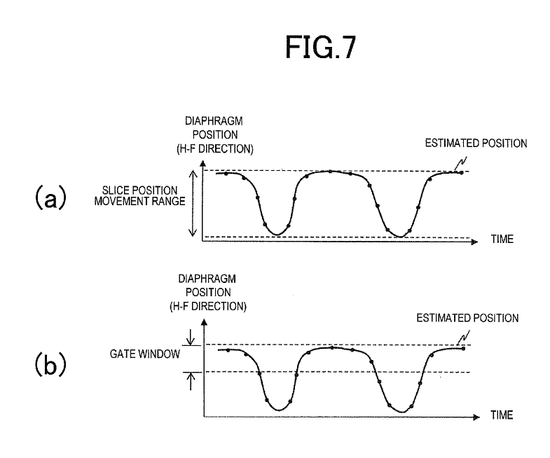

When correcting the slice position, as shown in FIG. 7(a), it can be calculated from the association information 601 how long (mm) or how many pixels the position zi in the H-F direction, which is estimated during the continuation of the main imaging, deviates from the position zj in the H-F direction, which is estimated from the position in the A-P direction at the time of scanogram imaging that has been used to determine the slice position. Therefore, this deviation is set as the amount of slice position correction (Δz=zj−zi), and the slice position is corrected by the amount of correction Δz at every repetition of imaging, thereby executing the pulse sequence.

On the other hand, when performing the gating, signals are collected when a position in the H-F direction estimated from the body motion position detected by the pressure sensor is in the range of a gate window GW set in the H-F direction, as shown in FIG. 7(b). Signal collection at a position deviating from the gate window GW is not performed. The slice position correction or the gating that is to be performed can be appropriately selected according to the imaging target or the imaging purpose.

By such main imaging, it is possible to acquire an image in which there is no influence of body motion. The acquired image is displayed on the display unit 108 together with other necessary pieces of information, for example, information regarding an object or imaging conditions (display control unit 1113).

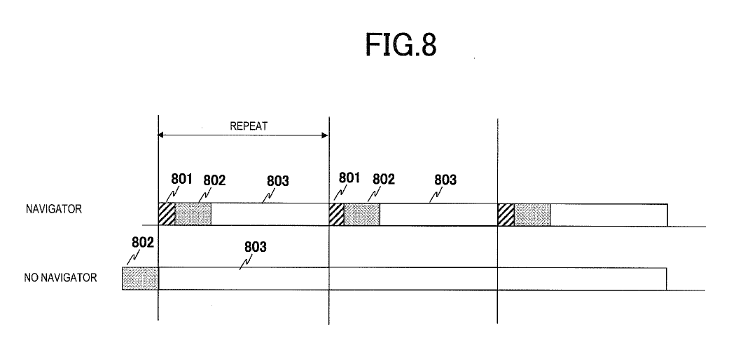

According to the present embodiment, during the main imaging, information from only the external monitor is used, and the navigator sequence affecting the imaging is not used. Therefore, it is possible to prevent an increase in the imaging time due to inserting the navigator sequence or the influence on the pulse sequence due to navigator echoes. For example, in the case of cine imaging of the heart to continuously capture the image in each phase in the cardiac cycle, a steady state free precession (SSFP) sequence for collecting echoes in the steady state is used in many cases. Therefore, as shown in FIG. 8, in order to create an SSFP state, a so-called idle application sequence 802 for applying the RF pulse without collecting echo signals is required before a main imaging sequence 803.

On the other hand, since the position of the heart is susceptible to respiratory motion, it is preferable to perform body motion control. Therefore, as shown in the diagram, when a navigator sequence 801 is added, the navigator sequence 801, which is performed whenever imaging is repeated, and the idle application sequence 802 for returning to the SSFP state that has collapsed due to the navigator sequence 801 are required. As a result, since it is not possible to acquire an image in the cardiac phase corresponding to these sequence execution times, an incomplete cine image is acquired. In contrast, when the present embodiment is applied, it is possible to acquire the information of the navigator without performing the navigator sequence. Therefore, as shown on the lower side in FIG. 8, it is possible to perform the idle application sequence 802 only once at the beginning and perform the SSFP sequence 803 continuously thereafter. Thus, it is possible to acquire images in all cardiac phases while eliminating the influence of the body motion as much as possible.

In addition, according to the present embodiment, it is possible to estimate a movement in a direction, which is difficult to detect with an external monitor among a plurality of body motion monitors, from the association information. Also for imaging in which a body motion in the estimated direction needs to be suppressed, it is possible to acquire a good image with only an external monitor.

In addition, in the above embodiment, the case has been described in which the body motion in the A-P direction is detected using the pressure sensor that is an external monitor, the body motion in the H-F direction is measured by the navigator sequence, and association information between both of the body motions is calculated. However, when a slice selection direction is the A-P direction (imaging of the COR plane), it is possible to detect the body motion in the A-P direction, which is the same direction as a pressure sensor, using a navigator and to acquire association information between both of the body motions. That is, the directions of movements detected by the external monitor and the internal monitor may be the same. Also in this case, a navigator sequence is not required during the main imaging, and it is possible to perform control using the position information in units of mm or a pixel acquired by the navigator sequence.

In addition, in the navigator sequence, it is possible to select the excitation region in any direction, such as the A-P direction, the H-F direction, or the R-L direction. If there is an image as an index in a region that is selected and excited, it is possible to detect displacement in any direction. Therefore, by acquiring the displacement in a plurality of arbitrary directions using the navigator sequence and calculating the relationship between the displacement in each direction and the displacement detected by the pressure sensor, it is possible to estimate the displacement of the cross section on the imaging section in any direction. Thus, it is possible to perform slice position correction or gating.

### Second Embodiment

The present embodiment is the same as the first embodiment in that the association between the position information from the external monitor, such as a pressure sensor, and the position information from the navigator sequence is performed and imaging control is performed using the association information during the main imaging. The present embodiment is characterized in that an association information update function is given. That is, an MRI apparatus of the present embodiment includes a storage unit that stores association information created by a body motion processing unit, and the body motion processing unit updates the association information stored in the storage unit using body motion information newly acquired from at least one of a plurality of body motion monitors.

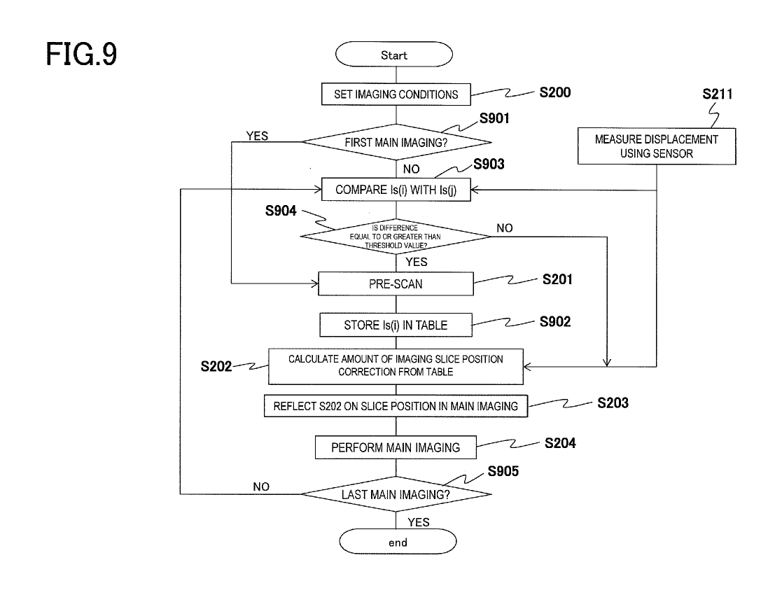

FIG. 9 shows the procedure of the second embodiment. In FIG. 9, steps of the same processing contents as the steps in FIG. 2 are denoted by the same reference numerals. First, in the case of first main imaging (determination step S901), the pre-scan step S201 is performed while performing the displacement measurement S211 using an external monitor (for example, a pressure sensor or bellows). In the pre-scan step S201, as shown in FIG. 3, navigator measurement is continuously performed, and time-series position information (that is, respiratory displacement) is acquired from the acquired navigator echo. A table is created by calculating the relationship between the respiratory displacement In acquired from the navigator echo and the respiratory displacement Is(i) measured by the external monitor. In addition, in the present embodiment, the respiratory displacement Is(i) measured by the external monitor at the time of a pre-scan is stored in the storage unit (S902).

In main imaging after the pre-scan S201, the amount of correction of the imaging slice position is calculated using the body motion position detected by the external monitor and the table of the association information of the displacement created in the pre-scan step S201 (S202), the slice position in the main imaging is corrected with the amount of correction (S203), and the main imaging is performed (S204). When continuing the imaging for the same object, the process returns to step S901, and the displacement Is(j) measured by the external monitor up to that point in time is compared with the displacement Is(i) measured during the execution of the pre-scan that is stored in the storage unit (S903). When the difference between both displacements (Is(i) and Is(j)) is equal to or greater than a threshold value set in advance (determination step S904), the pre-scan step S201 is performed again.

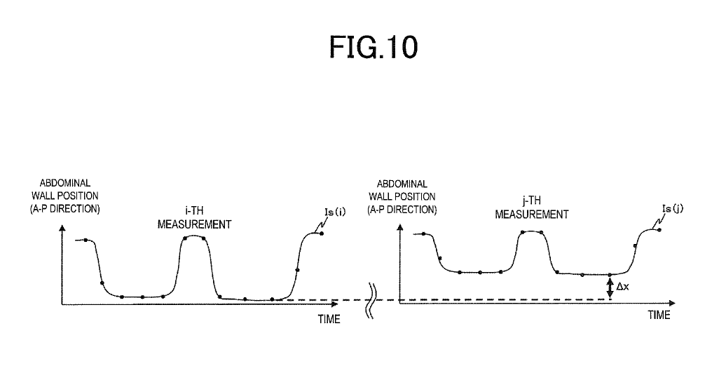

FIG. 10 shows an example of the displacement Is(i) measured at the time of the pre-scan (S311) and the displacement Is(j) measured during the repetition of the main imaging (S211), which are compared in step S903. In the example shown in the diagram, the amplitude of respiratory displacement is reduced during the repetition of the main imaging compared with that at the time of the pre-scan. In steps S903 and S904, for example, the amplitude of each displacement is calculated, and a difference Δx between the amplitudes is compared with a threshold value. Although the threshold value can be set arbitrarily, it is possible to use a slice thickness, for example.

In addition, when the gate window is set, the gate window width may be set as a threshold value. That is, when a shift in an amount corresponding to the slice thickness or the gate window width occurs in the displacement during the main imaging for the displacement at the time of a scan, it is determined that using the table created in the first pre-scan continuously is not appropriate. Therefore, a pre-scan is performed again to re-create a table of displacement association information. The method of calculating the displacement association information is the same as that described in the first embodiment. In the slice position correction amount calculation step S202 of the main imaging, the amount of slice correction is calculated using a new table.

On the other hand, when the difference between the displacements compared in the determination step S903 is less than the threshold value, processing of the slice position correction amount calculation step S202 is performed using the same table as in the previous imaging without performing the pre-scan. Then, main imaging reflecting the amount of correction calculated in step S202 is performed (S203 and S204), in the same manner as the first main imaging. Then, the steps S901 to S204 are repeated until the main imaging ends (determination step S905), and the pre-scan S202 is performed only when the deviation from the displacement measured at the time of previous imaging exceeds a threshold value.

In addition, although the case where the slice position correction of the main imaging is performed using the association information (table) between the displacement Is measured by the body motion sensor and the displacement In measured by the navigator is shown in FIG. 9, it is also possible to perform gating imaging using a table instead of the slice position correction.

According to the present embodiment, body motion information recorded at the time of a pre-scan is compared with body motion information acquired during the main imaging, and association information is re-acquired to use updated association information when the difference exceeds a predetermined range. Therefore, in response to a change in the respiratory status of the object during imaging, it is possible to perform the slice position correction or the gating imaging using the latest association information at all times. As a result, it is possible to improve the effectiveness of the present invention.

By storing the table of association information for each object, the present embodiment can also be applied when examining the same object at different dates and times. In this case, the first imaging in the flowchart of FIG. 9 may be replaced with first imaging for the object. If there is no change in the displacement measurement result of an external monitor, it is possible to omit a pre-scan in subsequent imaging. In this case, it is sufficient to perform only the main imaging using only an external monitor.

### Third Embodiment

In the first embodiment, the case has been described in which the position of the direction measured by the navigator sequence is estimated from the association information of the body motion and the slice correction or gating is performed for the direction estimated in the main imaging. However, the present embodiment is characterized in that slice correction in two or more directions is performed using both the estimated position and the position measured by the external monitor. That is, in an MRI apparatus of the present embodiment, a plurality of body motion monitors include body motion monitors that detect body motion information corresponding to different movement directions, and a control unit controls an imaging unit using a plurality of pieces of body motion information corresponding to different movement directions.

The procedure of the present embodiment is almost the same as the procedure of the first embodiment shown in FIG. 2. However, the present embodiment is different from the first embodiment in that the step S202 of calculating the amount of imaging slice position correction includes a step of calculating the amount of imaging slice position correction in a first direction using a position estimated from the displacement association information (table) and a step of calculating the amount of imaging slice position correction in a second direction (detection direction of an external monitor) using a position detected by an external monitor.

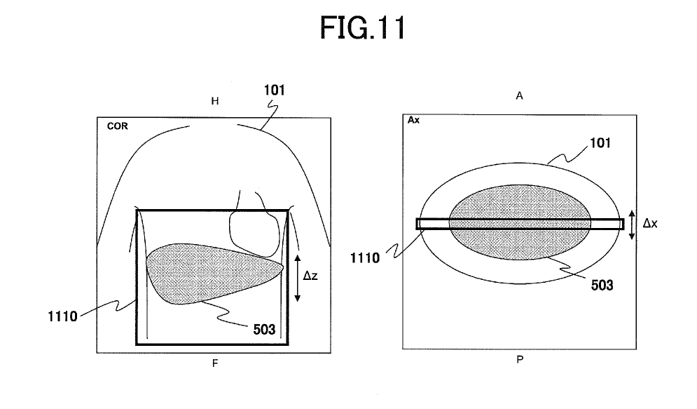

FIG. 11 shows an example of performing correction in the A-P direction and the H-F direction as first and second directions. FIG. 11 shows a case where a liver 1100 of an object is imaged on the COR plane. In this diagram, the left side shows the COR plane of a slice 1110, and the right side shows the position of the slice in the A-P direction (slice selection direction). This slice includes movements in both the H-F direction and the A-P direction due to respiratory motion. Although the slice selection direction is different from that in the first embodiment (FIG. 5) herein, the H-F direction is defined as a z direction and the A-P direction is defined as an x direction according to the definition in the first embodiment. In the correction amount calculation step S202, the position zi in the H-F direction is estimated from the position xi in the A-P direction detected by the pressure sensor and the table created in the pre-scan S201, and the amount of slice position correction Δz in the H-F direction is calculated using the estimated position zi and the amount of slice position correction Δx in the A-P direction is calculated using the position xi in the A-P direction detected by the pressure sensor.

Slice position adjustment can be realized, for example, by adjusting the irradiation frequency for the A-P direction and by adjusting the reception frequency for the H-F direction with this direction as a frequency encoding direction.

According to the present embodiment, a slice position is corrected for a plurality of directions using not only the estimated displacement but also the measured displacement. Therefore, it is possible to perform more exact slice position correction.

In addition, also in the present embodiment, a table created after a pre-scan may be updated in response to a change in the body motion amplitude during imaging by applying the second embodiment. In addition, instead of the slice position correction, application to gating imaging using the displacement information is also possible.

### Fourth Embodiment

The present embodiment is characterized in that a plurality of pieces of body motion information corresponding to different positions are acquired in the navigator sequence of the pre-scan S201. That is, in an MRI apparatus of the present embodiment, an internal monitor detects a plurality of pieces of body motion information, and a body motion processing unit creates a plurality of pieces of association information by associating each of the plurality of pieces of body motion information detected by the internal monitor with body motion information detected by an external monitor. The internal monitor can detect body motion information corresponding to different body motion detection positions as a plurality of pieces of body motion information. Alternatively, as a plurality of pieces of body motion information, it is possible to detect body motion information corresponding to different movement directions.

The procedure of the present embodiment is almost the same as the procedure of the first embodiment shown in FIG. 2. In the present embodiment, however, in the pre-scan step S201, the excitation region of the navigator sequence is changed to acquire body motion information (displacement) In1, In2, . . . , Ink from a plurality of regions. A plurality of tables (k tables) are created by associating the body motion information acquired from the plurality of regions with the body motion information Is from the body motion sensor detected in parallel with the navigator sequence.

In the main imaging (S202 and S203), using the association information of a region where the position of a slice to be imaged is included or a region closest thereto among the plurality of regions where the body motion information In1, In2, . . . , Ink is acquired, the slice position is corrected.

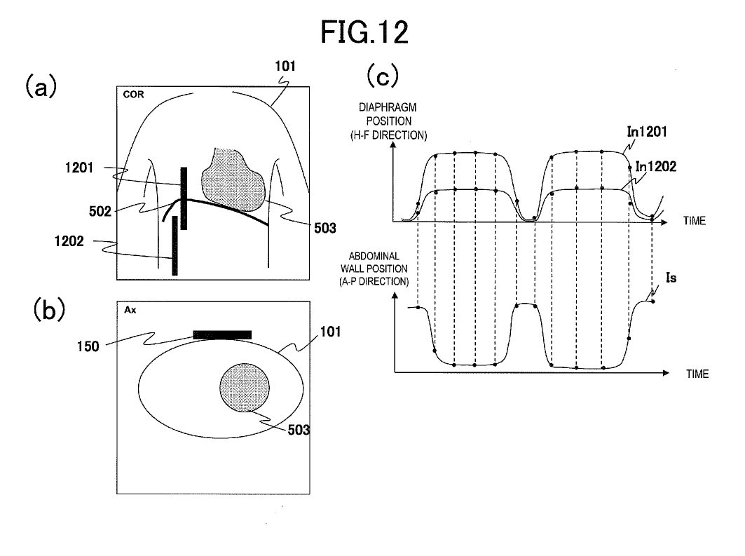

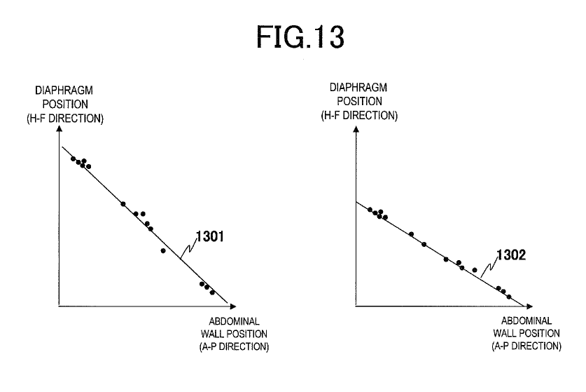

FIG. 12 shows an example when the present embodiment is applied to the imaging of the axial plane. FIG. 12(a) is a COR plane including the diaphragm 502 and the heart 503 of the object 101, and shows regions 1201 and 1202 excited by the navigator sequence. In the diagram, only two regions are shown. However, three or more regions may be present. For the regions 1201 and 1202, displacements In 1201 and In 1202 are detected from the positional change of the profile (top view in FIG. 12(c)). This displacement may be a displacement of the organ that is a predetermined marker included in a region, or may be calculated as an average value of the entire region. In parallel with the navigator acquisition of each region, the displacement Is is acquired from the external monitor 150 (bottom view in FIG. 12(c)), and each displacement detected by the navigator sequence and the displacement detected by the external monitor 150 are associated with each other. The method of association is the same as that described in the first embodiment. Thus, as shown in FIG. 13, association information (table) 1301 and 1302 of the same number as the number of displacements detected by the navigator is created.

In the main imaging, for example, it is assumed that an axial plane (FIG. 12 (b)) perpendicular to the COR plane is a slice surface and a plurality of slices are imaged in a range indicated by the arrow in (a). Then, when a slice position is the position of the region 1201, the amount of slice position correction is calculated using the table 1301 and the position detected by the external monitor at that time, and this is reflected in the main imaging. In addition, when the slice position has moved to the position of the region 1202, the amount of slice position correction is calculated using the table 1302 and the position detected by the external monitor at that time, and this is reflected in the main imaging. As shown in FIG. 12(a), when the regions 1201 and 1202 partially overlap, if the slice position is included in the overlap position, one of the tables may be used, or the average value of the amounts of correction calculated using both of the tables may be used as the amount of correction.

In addition, when the displacement detected by the navigator is the displacement of a predetermined marker in a region such as a diaphragm, the amount of slice position correction is calculated using a table created for a region including a marker closest to the slice position.

According to the present embodiment, it is possible to perform more accurate position correction. The present embodiment is suitable when imaging a relatively wide region.

### Modification Examples

In each of the embodiments described above, the case has been described in which the pressure sensor (external monitor) mounted on the object and the navigator sequence (internal monitor), which detects the body motion from the NMR signal, are used as a plurality of body motion monitors. However, various combinations are possible as a plurality of body motion monitors. As examples, (1) a combination of a plurality of kinds of external monitors that detect movements in different directions (for example, a pressure sensor and a three-dimensional position detector), (2) a plurality of kinds of external monitors and a navigator sequence of one direction (in this case, directions of the movements to be detected may be the same or different), and (3) one external monitor and navigator sequences of two directions can be mentioned.

While the embodiments of the present invention have been described, the present invention is not limited to these embodiments, and the features of the present invention included in the embodiments can be applied to the MRI apparatus and method independently or in combination. Main features of the present invention are as follows.

Position information of a plurality of body motion monitors is used. Therefore, since it is possible to detect movements in a plurality of directions of the body motion, it is possible to respond to an arbitrary imaging section. That is, when a plurality of body motion monitors detect movements in different directions, imaging can be controlled using the body motion information from the body motion monitor, which detects a movement in a direction corresponding to the slice direction, according to the imaging section.

Association information, in which the position information (displacement) of a plurality of body motion monitors is associated with each other in advance, is created. In this case, during imaging, body motion information is acquired from only one of a plurality of body motion monitors, and position information acquired by the other body motion monitors can be estimated based on the association information. Accordingly, it is possible to perform body motion control in the imaging of an arbitrary slice.

One of a plurality of body motion monitors is an internal monitor that measures the body motion using an NMR signal. The internal monitor is a navigator sequence, for example. The internal monitor can acquire the body motion in any direction according to the selection of the region to acquire a signal. Accordingly, the degree of freedom of the imaging section is high. By associating the body motion information of the internal monitor with the body motion information acquired from the other body motion monitors, it is possible to estimate the position detection result of the internal monitor without performing body motion detection by the internal monitor that affects imaging during the imaging. Therefore, it is possible to perform control that is versatile as the body motion control using the internal monitor.

In addition, in the main imaging, no internal monitor is used. Accordingly, it is possible to prevent an increase in the imaging time due to the navigator sequence, which is an internal monitor, or the like. As a result, the state (SSFP) of the spins that should be maintained in imaging using an internal monitor or the like is not affected.

### INDUSTRIAL APPLICABILITY

The present invention can acquire an image, in which there is no influence of body motion, accurately and easily in the MRI examination that is easily influenced by the body motion.

### REFERENCE SIGNS LIST

102: magnet (static magnetic field generation unit)
103: gradient magnetic field coil (gradient magnetic field generation unit)
109: gradient magnetic field power source (gradient magnetic field generation unit)
104: RF coil (high-frequency magnetic field generation unit)
110: RF transmission unit (high-frequency magnetic field generation unit)
105: RF probe (signal receiving unit)
106: signal detection unit (signal receiving unit)
107: signal processing unit
108: display unit
111: control unit
113: storage unit
115: body motion processing unit
150: pressure sensor (external monitor)
801: navigator sequence (internal monitor)

1. A magnetic resonance imaging apparatus, comprising:
an imaging unit including a static magnetic field magnet, a gradient magnetic field generation unit, a high-frequency magnetic field transmission unit, and a nuclear magnetic resonance signal receiving unit;
a signal processing unit that performs processing including image reconstruction using a nuclear magnetic resonance signal received by the nuclear magnetic resonance signal receiving unit;
a body motion processing unit that receives a plurality of pieces of body motion information from a plurality of body motion monitors that monitor a body motion of an object to be examined and associates a plurality of pieces of body motion information detected by the plurality of body motion monitors; and
a control unit that controls the imaging unit using body motion information detected by one of the plurality of body motion monitors and association information calculated by the body motion processing unit.

2. The magnetic resonance imaging apparatus according to claim 1,
wherein the control unit estimates body motion information of at least one of body motion monitors other than one body motion monitor using the body motion information detected by the one body motion monitor of the plurality of body motion monitors and the association information calculated by the body motion processing unit, and controls the imaging unit using the estimated body motion information.

3. The magnetic resonance imaging apparatus according to claim 1,
wherein at least one of the plurality of body motion monitors is an internal monitor that detects a body motion using the nuclear magnetic resonance signal received by the nuclear magnetic resonance signal receiving unit.

4. The magnetic resonance imaging apparatus according to claim 1,
wherein at least one of the plurality of body motion monitors is an external monitor that detects a movement of the object to be examined using a physical method.

5. The magnetic resonance imaging apparatus according to claim 1,
wherein the plurality of body motion monitors includes an internal monitor that detects a body motion using the nuclear magnetic resonance signal received by the nuclear magnetic resonance signal receiving unit and an external monitor that detects a movement of the object to be examined using a physical method.

6. The magnetic resonance imaging apparatus according to claim 5,
wherein the internal monitor and the external monitor detect movements in different directions.

7. The magnetic resonance imaging apparatus according to claim 5,
wherein the internal monitor and the external monitor detect movements in the same direction.

8. The magnetic resonance imaging apparatus according to claim 5,
wherein the internal monitor detects a plurality of pieces of body motion information, and
the body motion processing unit creates a plurality of pieces of association information by associating each of a plurality of pieces of body motion information detected by the internal monitor with body motion information detected by the external monitor.

9. The magnetic resonance imaging apparatus according to claim 8,
wherein the internal monitor detects body motion information corresponding to different body motion detection positions as the plurality of pieces of body motion information.

10. The magnetic resonance imaging apparatus according to claim 8,
wherein the internal monitor detects body motion information corresponding to different movement directions as the plurality of pieces of body motion information.

11. The magnetic resonance imaging apparatus according to claim 1,
wherein the plurality of body motion monitors includes body motion monitors that detect body motion information corresponding to different movement directions, and the control unit controls the imaging unit using a plurality of pieces of body motion information corresponding to different directions.

12. The magnetic resonance imaging apparatus according to claim 1, further comprising:
a storage unit that stores the association information created by the body motion processing unit,
wherein the body motion processing unit updates the association information stored in the storage unit using body motion information newly acquired from at least one of the plurality of body motion monitors.

13. The magnetic resonance imaging apparatus according to claim 1,
wherein the control unit controls the imaging unit to perform imaging by changing an imaging position of the object to be examined based on the body motion information received from the body motion monitors.

14. The magnetic resonance imaging apparatus according to claim 1,
wherein the control unit controls the imaging unit to perform imaging in a body motion range set in advance based on the body motion information received from the body motion monitors.

15. A magnetic resonance imaging method of performing imaging in accordance with body motion of an object to be examined, comprising:
a step of acquiring a plurality of pieces of body motion information from a plurality of body motion monitors;
a step of storing association information obtained by associating the plurality of pieces of body motion information acquired from the plurality of body motion monitors;
a step of acquiring body motion information from at least one of the plurality of body motion monitors and of estimating body motion information of body motion monitors different from the body motion monitor from which the body motion information has been acquired; and
a step of performing imaging using the estimated body motion information.
